(19) United States
(12) Patent Application Publication
(10) Pub. No.: US 2019/0192838 A1
(43) Pub. Date: Jun. 27, 2019

(54) FORM FITTING FOOT COVERING

(71) Applicant: Maud Lemercier, Lake Forest, IL (US)

(72) Inventor: Maud Lemercier, Lake Forest, IL (US)

(21) Appl. No.: 15/854,106

(22) Filed: Dec. 26, 2017

(51) Int. Cl. A61M 35/00; A41B 11/00; A43B 7/00; A61F 13/06

(52) U.S. Cl. CPC A61M 35/00; A41B 11/008; A41B 11/004; A61F 13/064; A43B 7/00

(57) ABSTRACT

A form fitting foot covering is disclosed and claimed. The foot covering includes an elongated tubular body constructed of a flex material, and includes an opening at one end that is adapted to allow a user to pull the foot covering over her foot. Five toes are disposed on an end of the elongated tubular body, and are adapted to allow a user to insert her toes therein. In certain embodiments, the foot covering includes a topical treatment predisposed within as well as a non-slip pattern on the bottom of the foot covering.

### FIELD OF THE DISCLOSURE

The present disclosure concerns foot covers and more particularly, form fitting foot covers that are intended for delivery of health or beauty related cremes or form fitting foot covers that include a non-slip exterior surface disposed on the portion covering the bottom of the wearer's foot.

### DESCRIPTION OF BACKGROUND

Disposable hand coverings made of latex and other elastic materials, such as nitrile, are used in in health care providers' offices throughout the world. They provide a simple way for a healthcare provider to handle biological materials without the provider experiencing skin-to-skin contact with the biological material. Similarly, disposable overshoes have also been used in hospitals and manufacturing clean rooms to guarantee that external contaminants are not able to cause harm to a subject.

In addition, foot coverings have been used to provide, for example, phototherapy or to deliver certain treatments. For example, when receiving phototherapy, various parts of a recipient's body can be covered by, for example, hand coverings and foot coverings. Similarly, in cases of burns or degloving, a loose-fitting foot covering can be used to deliver medication to the skin of a patient's foot in a healthcare setting.

However, the prior art has not addressed a number of situations. First, the foot coverings of the prior art tend to be loose fitting and unattractive; for example, a foot covering adapted for use by a burn victim for delivery of medicine will be intentionally loose fitting so as not to aggravate any wounds on the wearer's feet. Accordingly, for fashion and beauty conscious persons, such foot coverings are entirely unsuitable. Second, prior art foot coverings are not adapted to be used for normal ambulation without wearing shoes. Rather, prior art foot coverings are adapted to cover shoes, or to deliver a medicine or treatment to a patient.

### OBJECTS OF THE DISCLOSED SYSTEM, METHOD, AND APPARATUS

Accordingly, it is an object of the disclosure to provide a form fitting foot covering constructed of a stretch material.

Another object of the disclosure is to provide a form fitting foot covering adapted to deliver a beauty cream.

Another object of the disclosure is to provide a form fitting foot covering adapted for everyday use.

Another object of the disclosure is to provide a form fitting foot covering for use by patients in a hospital or clinic to provide a sterile foot covering.

Another object of the disclosure is to provide a form fitting foot covering for use in a nail spa, hotel, or hotel spa.

Another object of the disclosure is to provide a foot covering for use by passengers on airplanes.

Another object of the disclosure is to provide a foot covering for use at a health club or gymnasium.

Another object of the disclosure is to provide a foot covering for use while swimming.

Another object of the disclosure is to provide a foot covering for use at a public bath or shower.

Another object of the disclosure is to provide a foot covering for use in a sterile environment, such as a retirement home or a commercial kitchen.

Another object of the disclosure is to provide a foot covering for use by men, women, and children.

Other advantages of this disclosure will be clear to a person of ordinary skill in the art. It should be understood, however, that a system or method could practice the disclosure while not achieving all of the enumerated advantages, and that the protected disclosure is defined by the claims.

### SUMMARY OF THE DISCLOSURE

Generally speaking, pursuant to the various embodiments, the present disclosure provides a form fitting foot covering constructed of a flex material. The disclosed form fitting foot covering comprises an elongated tubular body constructed of a flex material. The elongated tubular body includes an opening that is adapted to be stretched by a user to accommodate the user's foot as she places the foot covering on her foot. In certain embodiments, the elongated tubular body can comprise a bottom surface having a non-slip tread pattern on the exterior of the bottom surface. In addition, the flexible foot covering includes five toes, each of which is adapted to allow a user's toe to be inserted within. In certain embodiments, each of the five toes can comprise an interior where a topical treatment, such as a beauty cream can be disposed within the toes of the foot covering. Finally, the disclosed foot covering includes an angled heel portion.

Variations, such as the use of medical treatments, as well as various uses, such as for use at health spas, swimming pools, and other users, are discussed herein.

### BRIEF DESCRIPTION OF THE DRAWINGS

Although the characteristic features of this disclosure will be particularly pointed out in the claims, the invention itself, and the manner in which it may be made and used, may be better understood by referring to the following description taken in connection with the accompanying drawings forming a part hereof, wherein like reference numerals refer to like parts throughout the several views and in which:

FIG. 1 is a perspective view of a form fitting foot covering constructed in accordance with this disclosure.

FIG. 1a is a perspective view of an alternative form fitting foot covering constructed in accordance with this disclosure.

FIG. 2 is a perspective view of a package for holding form fitting foot coverings constructed in accordance with this disclosure.

FIG. 2a is a perspective view of a rolled-up foot covering constructed in accordance with this disclosure so as to be able to multiple pairs of foot coverings in a package.

FIG. 2b is a perspective view of a box for dispensing form fitting foot coverings constructed in accordance with this disclosure.

FIG. 3 is a perspective view of three form fitting foot coverings of different sizes constructed in accordance with this disclosure.

FIG. 3a is a perspective view of a three alternative form fitting foot coverings of different sizes constructed in accordance with this disclosure.

FIG. 3 is a perspective view of a form fitting foot covering after being taken out of the package and before being placed on a wearer's leg.

FIG. 4 is a perspective view of an alternative form fitting foot covering constructed in accordance with this disclosure including a non-slip exterior surface and disposed on a user's foot.

FIG. 4a is a perspective view of another alternative form fitting foot covering constructed in accordance with this disclosure including a non-slip exterior surface and disposed on a user's foot.

FIG. 5 is a perspective view of a form fitting foot covering constructed in accordance with this disclosure illustrating the placement of the foot covering on a wearer's foot.

FIG. 6 is a cutout view of a form fitting foot covering constructed in accordance with this disclosure illustrating the placement of beauty cream within the foot covering.

FIG. 7 is a perspective view of an alternative form fitting foot covering constructed in accordance with this disclosure.

FIG. 8 is a perspective view of a second alternative form fitting foot covering constructed in accordance with this disclosure.

A person of ordinary skills in the art will appreciate that elements of the figures above are illustrated for simplicity and clarity, and are not necessarily drawn to scale. The dimensions of some elements in the figures may have been exaggerated relative to other elements to help understanding of the present teachings. Furthermore, a particular order in which certain elements, parts, components, modules, steps, actions, events and/or processes are described or illustrated may not be actually required. A person of ordinary skills in the art will appreciate that, for the purpose of simplicity and clarity of illustration, some commonly known and well-understood elements that are useful and/or necessary in a commercially feasible embodiment may not be depicted in order to provide a clear view of various embodiments in accordance with the present teachings.

### DETAILED DESCRIPTION

This disclosure involves the use of a foot covering constructed out of a flex material. A flex material is one that can be easily deformed or manipulated so as to conform to a wearer's foot. Examples of flex materials include, but are not limited to latex, natural rubber latex, synthetic polyisoprene, polyurethane, silicone, nitrile, and other thermoplastic elastomers and polymers.

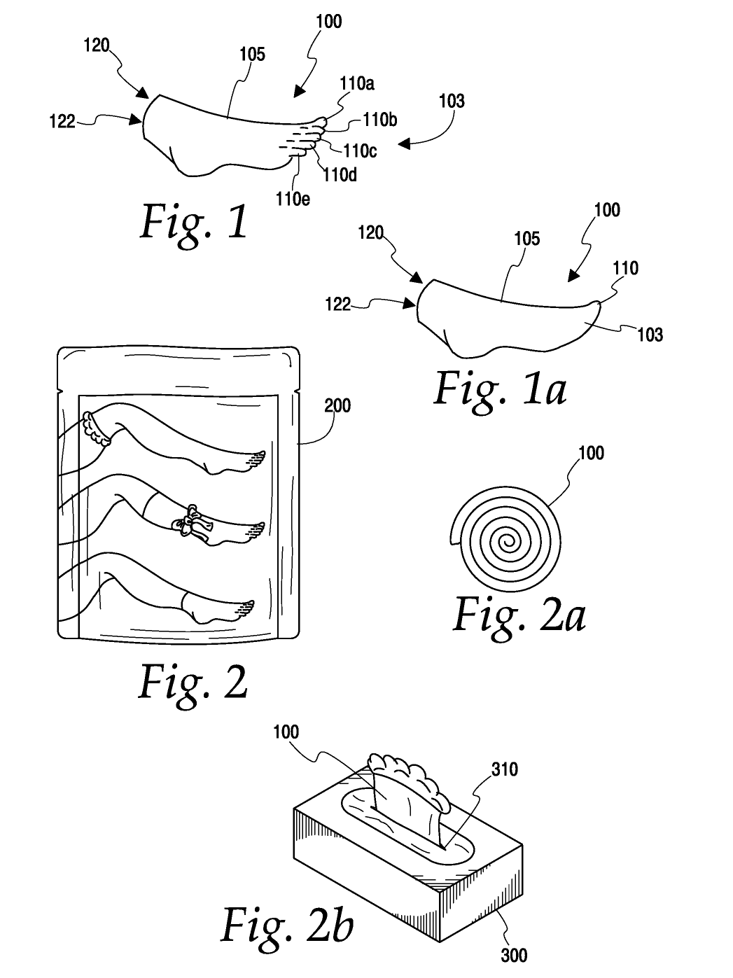

Turning to the Figures and to FIG. 1 in particular, a form fitting foot covering 100 is generally indicated and shown. The foot covering 100 includes a generally tubular body 105 having five toes 110a,110b,110c,110d,110e disposed at a distal first end 103. The foot covering 100 includes a near second end 120 that incorporates an opening 122 for receiving a person's foot. The covering 100 is formed in the general shape of a fitted sock wherein the body 105 is seamless having a substantially uniform width. A portion of the body 105 is angled to have a foot and ankle contour; i.e., to fit the form of a person's foot. The foot covering 100 is constructed of a flex material as described above. This allows the foot covering 100 to tightly conform to the wearer's foot and leg, and display the same in an aesthetically pleasing manner.

Turning to FIG. 1a, an alternative design is illustrated. The alternative form fitting foot covering 100 includes a generally tubular body 105. The tubular body 105 includes a distal first end 103 and a near second 120. The second end 120 incorporates an opening 122 for receiving a person's foot. The alternative form fitting foot covering is generally applicable for use, and especially well suited for use in, for example, a medical setting, or for a person who requires a covering to fit over a foot that is partially covered by a cast. In such a circumstance, a foot covering lacking individual toes is especially advantageous.

Turning to FIGS. 2 and 2a, the foot covering 100 can be stored quite compactly when, for example, rolled up as shown in FIG. 2a. This can make storing multiple sets of the foot covering 100 in a single package 200 possible, even when each foot covering 100 includes beauty cream as described further herein. Other, alternative packaging can also be used. For example, an airline may wish to provide the disclosed foot coverings to its business class and first class passengers, and may provide them with the collection of sundries that it provides such customers.

Turning to FIG. 2b, an alternative packaging and means for dispensing foot coverings is disclosed. In particular, a substantially rectangular box 300 is shown. The box 300 includes an opening 310 by which foot coverings 100 can be dispensed. Such a box can be used within a sterile location, such as a hospital, or it can be placed within a particular location, such as within the an easily accessible location of the first class or business class cabin of an airplane. A user can simply walk up to the box 300, and pull as many pairs of foot coverings 100 as are needed from the box 300 from the opening 310.

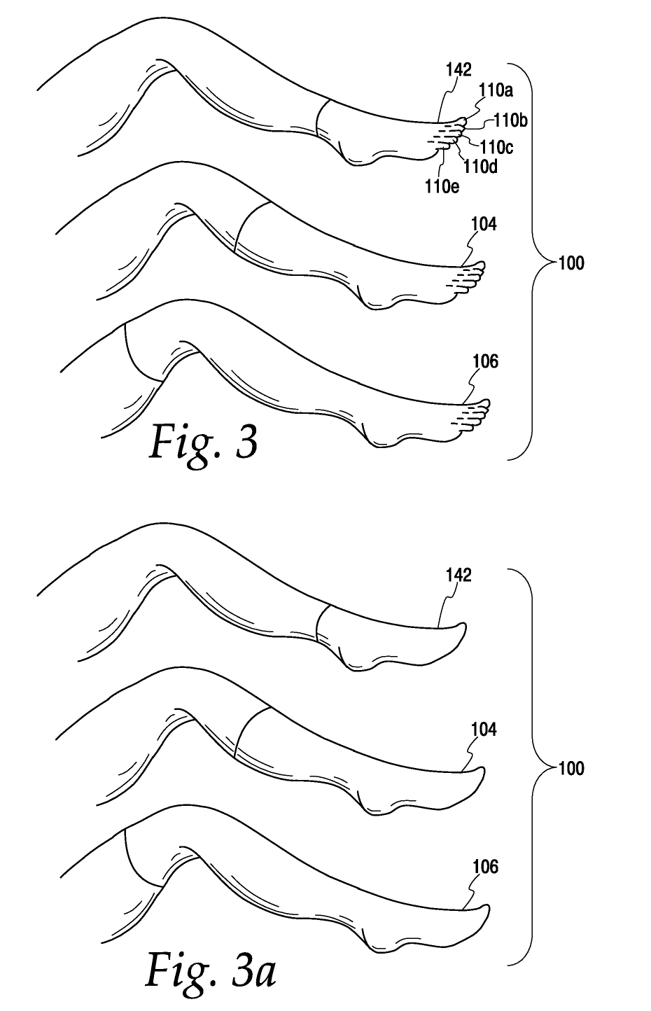

Turning to FIG. 3, three different sizes of form fitting foot coverings are displayed, and collectively referred to as 100. The first foot covering 102 is sized to cover a wearer's foot and ankle, the second foot covering 104 is sized to cover a wearer's leg through mid-calf, and a third foot covering 106 is sized to go over a wearer's knee.

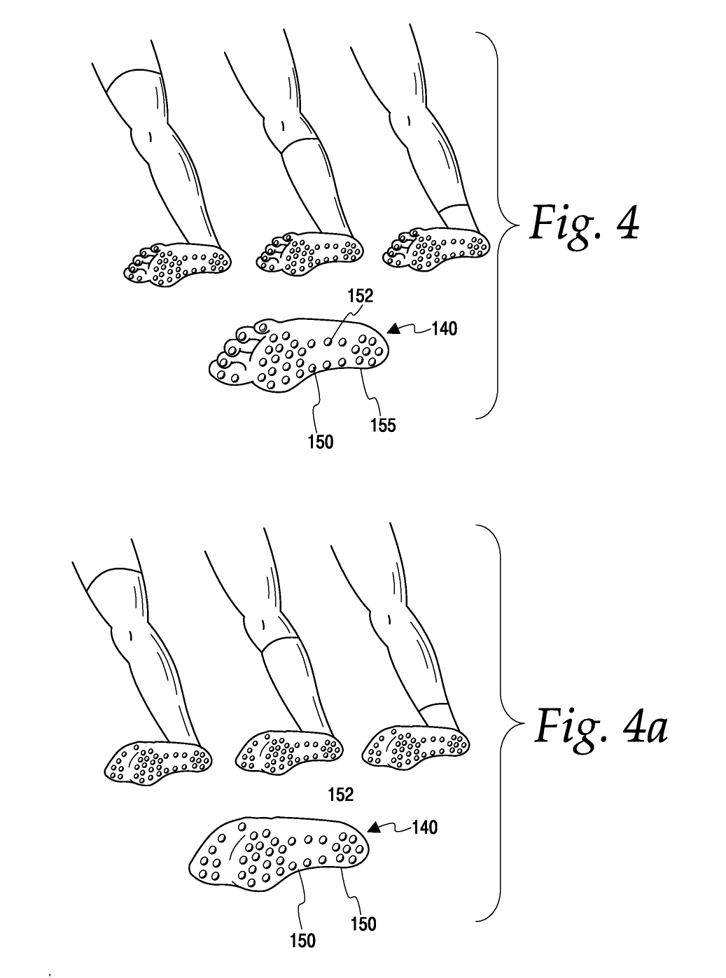

With reference to FIG. 4, an alternative form fitting foot covering 140 having a non-slip surface on its bottom surface 150 is shown. Generally, this variant of the foot covering 140 will include a thicker and software bottom surface 150 than other variants. In addition, the form fitting foot covering 140 includes a non-slip tread pattern 152, which in this case comprises a number of small bumps 155 spaced throughout the bottom surface 150 of the foot covering 140. In particular, the non-slip pattern 152 is adapted to provide a higher coefficient of friction, while maintaining a high level of surface area contact with the ground (when the foot covering is worn by a user and used as intended).

Turning to FIG. 4a, an alternative form fitting foot covering 140 having a non-slip surface on its bottom surface 150 is shown. As with the previous variant, this foot covering 140 will also include a thicker and software bottom surface 150 than other variants. In addition, the form fitting foot covering 140 includes a non-slip tread pattern 152, which can comprise a number of small bumps 155 spaced throughout the bottom surface 150 of the foot covering.

Turning to FIG. 5, a person putting on a form fitting foot covering constructed in accordance with this disclosure is illustrated. In particular, the user pulls the opening 122 open using the user's hands. The user then slides the foot covering 100 onto the user's foot, while manipulating the user's toes to fit into the toes 110a,110b,110c,110d,110e of the foot covering 100.

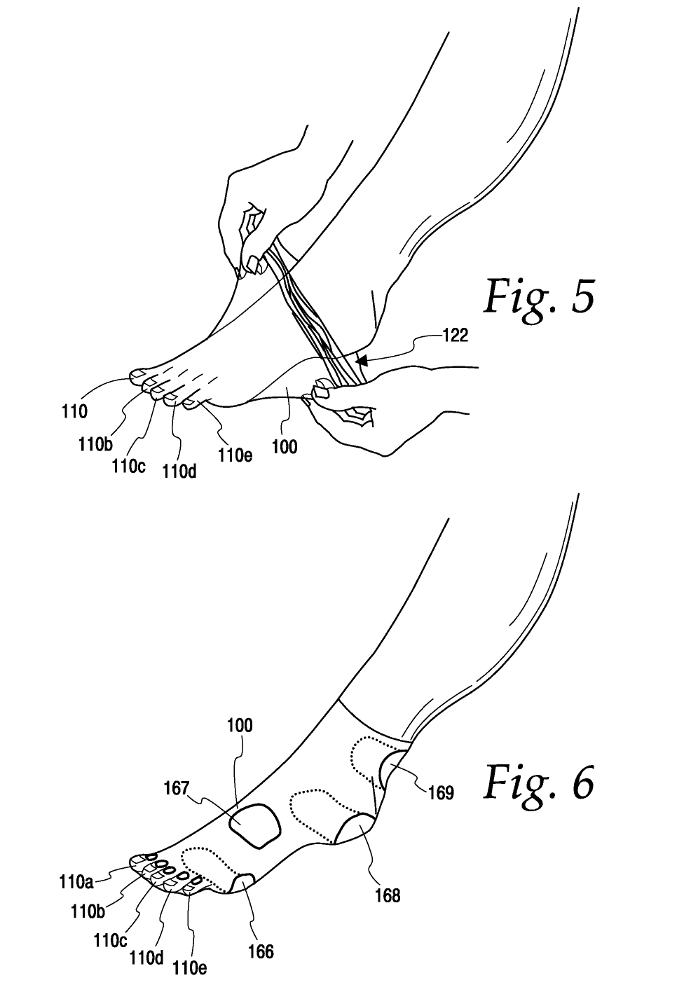

Turning to FIG. 6, a cutout view of a form fitting foot covering 100 constructed in accordance with this disclosure is shown. In particular, the cutout illustrates the placement of beauty cream, or some other type of topical agent, within the foot covering 100. Assuming that beauty cream is used it can be predisposed within the foot covering 100 so that it comes into contact with the parts of a user's foot that typically show issues. For example, beauty cream can be placed surrounding each toe in locations 165a,165b,165c,165d,165e, the ball of foot area 166, the top of the center of the user's foot 167, the heel area 168, and the ankle area 169. By providing beauty cream directly within the foot covering 100, it insures that the beauty cream is delivered to the proper areas of the wearer's foot; for example, the user can put the foot covering 100 on before going to sleep, and allow the beauty cream to work throughout the night. The foot covering 100 will maintain the beauty cream in place rather than having the same being subject to wiping off as the user brushes against coverings, etc.

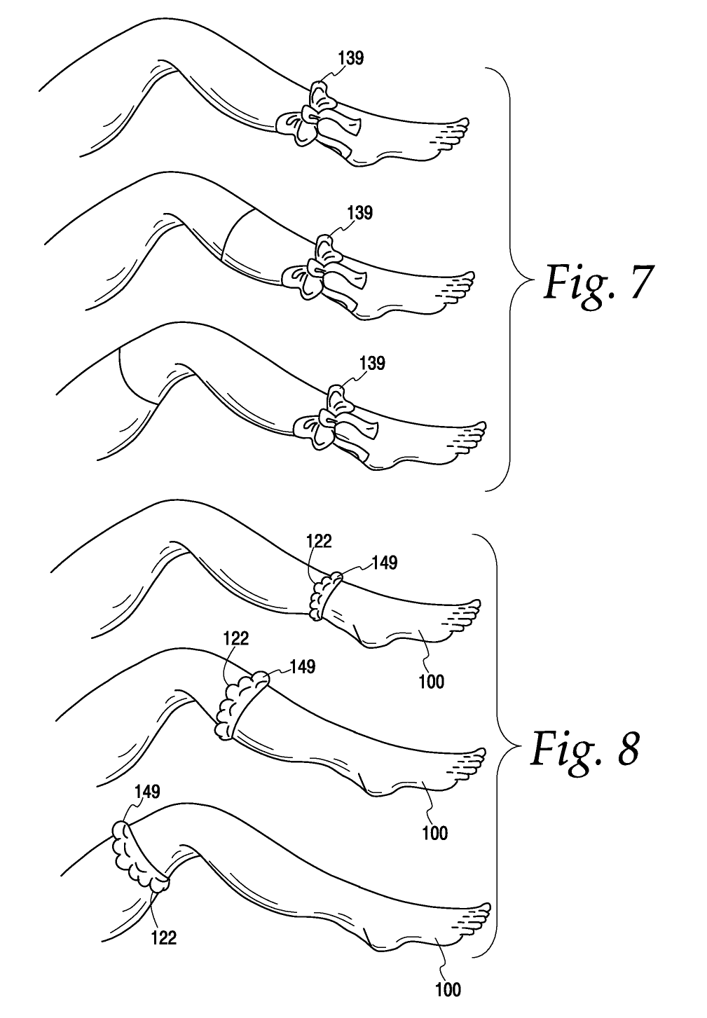

Turning to FIGS. 7 and 8, alternative designs for a form fitting foot covering are shown. In particular, these designs include, in the case of FIG. 7, a bow 139, and in the case of FIG. 8, faux lace 149 disposed near the opening 122 of the foot covering 100.

Similarly, the same mechanism can be used to deliver medicinal treatments. For example, a user that is being treated for eczema can have a medication, such as a steroid cream, delivered in a matter similar to that described above; i.e., by having the topical treatment predisposed within the foot covering so that it comes into contact with the area of the user's foot or ankle suffering from eczema. Other conditions that require topical treatment of medications can also be treated by predisposing the topical medicine within the foot covering.

The foregoing description of the disclosure has been presented for purposes of illustration and description, and is not intended to be exhaustive or to limit the disclosure to the precise form disclosed. The description was selected to best explain the principles of the present teachings and practical application of these principles to enable others skilled in the art to best utilize the disclosure in various embodiments and various modifications as are suited to the particular use contemplated. It should be recognized that the words “a” or “an” are intended to include both the singular and the plural. Conversely, any reference to plural elements shall, where appropriate, include the singular.

It is intended that the scope of the disclosure not be limited by the specification, but be defined by the claims set forth below. In addition, although narrow claims may be presented below, it should be recognized that the scope of this invention is much broader than presented by the claim(s). It is intended that broader claims will be submitted in one or more applications that claim the benefit of priority from this application. Insofar as the description above and the accompanying drawings disclose additional subject matter that is not within the scope of the claim or claims below, the additional inventions are not dedicated to the public and the right to file one or more applications to claim such additional inventions is reserved.

What is claimed is:

1. A form fitting foot covering comprising:
i) an elongated tubular body constructed of a flex material, the elongated tubular body being adapted to be stretched by a user to accommodate a user's foot;
ii) five toes disposed at a first end of the elongated tubular body, each of the five toes being closed, and adapted to fit over a wearer's toe;
iii) a second end having an opening adapted to allow insertion of a user's foot; and
iv) an angled heel portion.

2. The form fitting foot covering of claim 1 further comprising one or more deposits of cream disposed within an interior of the tubular body.

3. The form fitting foot covering of claim 2 wherein at least one deposit of cream is disposed in a toe of the foot covering.

4. The form fitting foot covering of claim 1 wherein the elongated tubular body further comprises a bottom surface, an exterior of the bottom surface having a non-slip tread pattern.

5. The form fitting foot covering of claim 1 wherein the non-slip tread pattern consists of a plurality of bump protrusions extending from the exterior of the bottom surface and being dispersed regularly along the bottom surface.

6. A form fitting foot covering comprising:
i) an elongated tubular body constructed of a flex material, the elongated tubular body being adapted to be stretched by a user to accommodate a user's foot;
ii) the elongated tubular body further comprising a bottom surface, the bottom surface having an exterior surface with a non-slip tread pattern;
iii) five toes disposed at a first end of the elongated tubular body, each of the five toes being closed and adapted to fit over a wearer's toe;
iv) each of the five toes comprising an interior, the interior having a deposit of beauty cream;
v) a second end having an opening adapted to allow insertion of a user's foot; and
vi) an angled heel portion.

7. A form fitting foot covering comprising:
i) an elongated tubular body constructed of a flex material, the elongated tubular body being adapted to be stretched by a user to accommodate a user's foot;
ii) a rounded first end located distally; and
iii) a second end having an opening adapted to allow insertion of a user's foot.

8. The form fitting foot covering of claim 7 further comprising one or more deposits of cream disposed within an interior of the tubular body.

9. The form fitting foot covering of claim 8 wherein at least one deposit of cream is disposed in a toe of the foot covering.

10. The form fitting foot covering of claim 7 wherein the elongated tubular body further comprises a bottom surface, an exterior of the bottom surface having a non-slip tread pattern.

11. The form fitting foot covering of claim 7 wherein the non-slip tread pattern consists of a plurality of bump protrusions extending from the exterior of the bottom surface and being dispersed regularly along the bottom surface.
